(19) United States
(12) Patent Application Publication
(10) Pub. No.: US 2004/0121961 A1
(43) Pub. Date: Jun. 24, 2004

(54) Uroguanylin and cyclooxygenase-2 inhibitor combinations for inhibition of intestinal cancer

(76) Inventor: Jaime Masferrer, Ballwin, MO (US)

Correspondence Address: Pharmacia Corporation, Corporate Patent Department, PO Box 1027, Chesterfield, MO, 63006, US

(21) Appl. No.: 10/467,145

(22) PCT Filed: Feb. 4, 2002
(86) PCT No.: PCT/US02/03201

(51) Int. Cl. A61K 38/10

(52) U.S. Cl. 514/15

(57) ABSTRACT

Disclosed is a method of retarding the development of polyps and prevention, inhibition and treatment of cancer in the intestine of a subject by administration of a composition comprising a peptide with the active domain of uroguanylin or any agonist peptide or compound binding to the guanylate cyclase receptor GC-C in the intestine in combination with a naturally occurring, derived from a naturally occurring, or a chemically synthesized cycloogenase-2 inhibitor, preferably a selective cyclooxygenase-2 inhibitor.

### BACKGROUND OF THE INVENTION

The present invention relates to the use of certain peptides, more particularly the use of uroguanylin and prouroguanylin in combination with any one of or combination of naturally occurring, extract of a naturally occurring, or a chemically synthesized cyclooxygenase-2 inhibitor, preferably a selective cyclooxygenase-2 inhibitor or inhibitors, to retard the development of polyps and prevent, inhibit or treat cancer in the intestine.

The pathogenesis of colorectal cancer is characterized as a multistep process that begins with increased proliferation and/or decreased apoptosis of colorectal epithelial cells resulting in generation of polyps, followed by adenoma formation and ultimately to adenocarcinoma. Certain individuals develop multiple colorectal adenomas and subsequent carcinomas early in life because of a genetic defect in the APC gene responsible for causing a condition called familial adenomatous polyposis (FAP). Dihlmann et al, Dominant negative effect of the APC 1309 mutation: a possible explanation for genotype-phenotype correlations in familial adenomatous polyposis, Cancer Res. 1999 Apr. 15, 59(8): 1857-60. Chemoprevention has evolved during the last decade as a viable strategy for cancer prevention, with the aim of controlling the development of cancer through pharmacological and/or dietary intervention prior to the appearance of a clinically detectable tumor. Reddy, B. S. (1997) Chemoprevention of colon cancer by dietary administration of naturally-occurring and related synthetic agents, Adv. Exp. Med. Biol. 400B:931-936.

Uroguanylin and guanylin are structurally related enteric peptide hormones that are secreted intraluminally by different types of cells, include enterochromaffin, goblet and others within the intestinal mucosal lining. A receptor for theses peptides that has been identified at the molecular level is a transmembrane form of guanylate cyclase (GC) known as GC-C. Krause, W. J. et al, The guanylin and uroguanylin peptide hormones and their receptors, Acta. Anat. (Basel) 160:213-231 (1997). GC-C receptors are localized on the luminal surface of enterocytes throughout the GI tract. Swenson, E. S. et al, The guanylin/STa receptor is expressed in crypts and apical epithelium throughout the mouse intestine, Biochem. Biophys. Res. Commun. 225:1009-1014 (1996). Binding of uroguanylin or guanylin to the extracellular domain of GC-C receptors stimulates intracellular production of the second messenger cGMP, resulting in activation of cystic fibrosis transmembrane conductance regulator (CFTR), the apical membrane channel for efflux of chloride from enterocytes lining the intestinal tract. Forte, L. R. et al, Salt and water homeostasis: uroguanylin is a circulating peptide hormone with naturiuretic activity, Am. J. Kidney Dis. 28:296-304 (1996). Activation of CFTR chloride channel proteins and the subsequent enhancement of transepithelial secretion of chloride leads to stimulation of sodium (Na⁺) and water secretion into the intestinal lumen. Forte, L. R. et al, Guanylin regulatory peptides: structures, biological activities mediated by cyclic GMP and pathobiology, Regul. Pept. 81:25-39 (1999). Therefore, one of the major physiological functions of these hormones is the regulation of fluid and electrolyte transport in the gastrointestinal (GI) tract by serving as paracrine regulators of CFTR activity.

The precursor of uroguanylin is prouroguanylin, which is broken down by endogenous proteases in the intestinal tract to produce the active uroguanylin. Chymotrypsin activates prouroguanylin to cleave it into its active form of uroguanylin. Forte, et el, Salt and Water Homeostasis: Uroguanylin Is a Circulating Peptide Hormone With Natriuretic Activity, Am. J. Kid. Dis. 1996, 28, No.2, 296-304. Uroguanylin is an acid-stable and proteolysis-resistant peptide, which will remain intact to act on the intestinal lumen directly rather than being absorbed systemically. Uroguanylin and guanylin are produced throughout the intestinal mucosa and in the myocardium. Forte et al, Salt and water homeostasis:uroguanylin is a circulating peptide hormone with natriuretic activity Am. J. Kidney Dis. 28:296-304 (1996). Human uroguanylin has been isolated from human urine and has been chemically synthesized by solid phase peptide synthesis as described in U.S. Pat. No. 5,489,670 for Human Uroguanylin.

Binding of uroguanylin or guanylin to the guanylin cyclase receptor stimulates the intracellular production of the cGMP ultimately resulting in the stimulation of salt and water secretion into the intestinal lumen. Uroguanylin and guanylin receptors are found on the luminal surface of epithelial cells lining the intestinal tract and renal proximal tubules as well as in other organs. Forte et al, Salt and Water Homeostasis: Uroguanylin Is a Circulating Peptide Hormone with Natriuretic Activity, Am. J. Kid. Dis.1996, 28, No. 2, 296-304. Uroguanylin has been found to stimulate increases in cyclic GMP levels in a manner similar to another family of heat stable enterotoxins (STs) secreted by pathogenic strains of E. coli and other enteric bacteria that activate intestinal guanylate cyclase and cause secretory diarrhea, which is a major cause of traveler's diarrhea and many deaths in developing countries. Forte et al, Lymphoguanylin: Cloning and Characterization of a Unique Member of the Guanylin Peptide Family, Endocrinology Vol. 140, No. 4, p.1800-1806. These ST peptides act as molecular mimics of the endogenous mammalian peptides of uroguanylin and prouroguanylin. Forte et al, Endocrinology Vol. 140, No. 4, p.1800. Unlike uroguanylin the STs from enteric bacteria do not have a decrease in potency when the pH changes in the colon. STs are more potent than either uroguanylin or guanylin under both acidic and alkaline conditions. Forte et al, Guanylin: a peptide regulator of epithelial transport, The FASEB Journal, vol. 9, 643-650 (1995). Uroguanylin is believed to regulate fluid and electrolyte transport in a manner similar to guanylin and the STs in the GI tract. Therefore, as mentioned in previous publications the human uroguanylin may act as a laxative and be useful in patient suffering from constipation.

Prostaglandins play a major role in the inflammation process and the inhibition of prostaglandin production, especially production of PGG₂, PGH₂ and PGE₂, has been a common target of anti-inflammatory drug discovery. However, common non-steroidal anti-inflammatory drugs (NSAID's) that are active in reducing the prostaglandin-induced pain and swelling associated with the inflammation process are also active in affecting other prostaglandin-regulated processes not associated with the inflammation process. Thus, use of high doses of most common NSAID's can produce severe side effects, including life threatening ulcers, that limit their therapeutic potential. An alternative to NSAID's is the use of corticosteroids, which also produce severe adverse effects, especially when long term therapy is involved.

NSAIDs have been found to prevent the production of prostaglandins by inhibiting enzymes in the human arachidonic acid/prostaglandin pathway, including the enzyme cyclooxygenase (COX). The recent discovery of an inducible enzyme associated with inflammation (named “cyclooxygenase-2 (COX-2)” or “prostaglandin G/H synthase II”) provides a viable target of inhibition which more effectively reduces inflammation and produces fewer and less drastic side effects.

Compounds that selectively inhibit cyclooxygenase-2 have also been described in the following individual publications.

U.S. Pat. No. 5,380,738.

U.S. Pat. No. 5,344,991.

U.S. Pat. No. 5,393,790.

U.S. Pat. No. 5,434,178.

U.S. Pat. No. 5,474,995.

U.S. Pat. No. 5,510,368.

WO 96/06840.

WO 96/03388.

WO 96/03387.

WO 96/19469.

WO 96/25405.

WO 95/15316.

WO 94/15932.

WO 94/27980.

WO 95/00501.

WO 94/13635.

WO 94/20480.

WO 94/26731.

Further, natural Cyclooxygenase-2 inhibitors have been disclosed in “Selective Cyclooxygenase-2 Inhibition from Edible Plant Extracts”, U.S. Non-provisional application Ser. No. 09/737,892, filed Jan. 3, 2001; “Selective Cyclooxygenase-2 Inhibition from Non-edible Plant Extracts”, U.S. Non-provisional application Ser. No. 09/737,701, filed Jan. 3, 2001; and “Selective Cyclooxygenase-2 Inhibition from Plant Extracts”, U.S. Non-provisional application Ser. No. 09/738,041, filed Jan. 3, 2001. [Pyrazol-1-yl]benzenesulfonamides have been described as inhibitors of cyclooxygenase-2 and have shown promise in the treatment of inflammation, arthritis, and pain, with minimal side effects in pre-clinical and clinical trials. Their use for preventing colon cancer has been described in U.S. Pat. No. 5,466,823. However, their use for treating or preventing intestinal cancer, in combination with uraguanylin, has not been described.

### SUMMARY OF THE INVENTION

The combination of certain peptides, particularly uroguanylin or prouroguanylin, with a single or multiple natural occurring, extract of a natural occurring, or a chemically synthesized cyclooxygenase-2 inhibitor, preferably a selective cyclooxygenase-2 inhibitor or inhibitors, may be useful in treating, preventing, inhibiting or retarding the development of polyps and cancer in the intestine.

Among the objects and features of the present invention may be noted the provision of a process for retarding the development of polyps and preventing, and a process for inhibiting and treating cancer or neoplasia in a subject. Preferably the method is useful for treating the development of polyps and preventing, and a process for inhibiting and treating cancer in the intestine of a subject, more preferably the small intestine or the colon.

Briefly, therefore, the present invention is directed to a process for retarding the development of polyps in a subject which comprises the administration of a peptide including the amino acid sequence:

Asp- Asp- Cys- X₁- X₂- Cys- X₃- Asn- X₄- X₅- Cys-
X₆- X₇- Cys

wherein each of X₁, X₂, X₃, X₄, X₅, X₆, and X₇ is an amino acid residue, and the polypeptide is cross-linked by a disulfide bond between the cystine residue immediately adjacent the amine group of X₁ and the cystine residue immediately adjacent the amine group of X₆ and by a disulfide bond between the cystine residue immediately adjacent the amine group of X₃ and the cystine residue immediately adjacent the carboxy group of X₇, in combination with any one of or combination of naturally occurring, or an extract of a natural occurring, or a chemically synthesized cyclooxygenase-2 inhibitor, preferably a selective cyclooxygenase-2 inhibitor or inhibitors.

US Patent application number PCT/US00/21998 (herein incorporated by reference) describes the use of uroguanylin as an intestinal cancer inhibiting agent.

The invention is further directed to a process for retarding the development of polyps and to a process for the prevention, inhibition or treatment of cancer in a subject by administration of a composition comprising any one of or combination of the following peptides: uroguanylin, human uroguanylin, pro-uroguanylin, and human pro-uroguanylin, guanylin, lymphoguanylin, prolymphoguanylin and heat stable enterotoxin in combination with any one of or combination of naturally occurring, or an extract of a natural occurring, or a chemically synthesized cyclooxygenase-2 inhibitor, preferably a selective cyclooxygenase-2 inhibitor or inhibitors.

Additionally, the invention is directed to a process for retarding the development of polyps and a process for the prevention, inhibition or treatment of cancer by administration of a composition comprising any one of or a combination of agonist peptides and/or other agonist compounds to the guanylate cyclase receptor GC-C in combination with any one of or combination of naturally occurring, or an extract of a natural occurring, or a chemically synthesized cyclooxygenase-2 inhibitor, preferably a selective cyclooxygenase-2 inhibitor or inhibitors.

The cancer or neoplasia which can be treated with the present inventive method can be located anywhere in the body, for example, the head, neck, chest, lungs, skin, liver, blood, kidneys, heart, intestines, bladder, gall bladder, brain, throat, musculoskeletal system, lymphatic system, central nervous system, and others. Preferably, the methods of the present invention are used to treat cancer or neoplasia located in the intestine, for example, the small intestine or colon.

Other objects of this invention will be in part apparent and, in part, pointed out hereinafter.

### DETAILED DESCRIPTION OF THE PREFERRED EMBODIMENTS

The term “treatment” includes partial or total inhibition of the tumor growth, either benign or malignant, spreading or metastasis, as well as partial or total destruction of the neoplastic cells.

The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia.

The phrase “therapeutically-effective” is intended to qualify the amount of each agent which will achieve the goal of improvement in disease severity and the frequency of incidence over treatment of each agent by itself, while avoiding adverse side effects typically associated with alternative therapies.

The term “subject” for purposes of treatment includes any human or animal subject who has any one of the known neoplasia or tumor disorders, and preferably is a human subject. For methods of prevention, the subject is any human or animal subject, and preferably is a human subject who is at risk for obtaining an intestinal cancer or neoplasia-related disorder, either benign or malignant, including metastasis. The subject may be at risk due to exposure to carcinogenic agents, being genetically predisposed to have the neoplasia, and the like.

The term neoplasia includes both benign and cancerous tumors and growths.

In the method above, the epithelial cell-derived neoplasia includes epithelial carcinomas such as basal cell carcinoma, adenocarcinoma, colon cancer, prostate cancer, renal cell carcinoma, and other known neoplasias that effect epithelial cells throughout the body. Preferably, the epithelial cell-derived neoplasia is selected from gastrointestinal cancer, liver cancer, prostate cancer, kidney cancer, brain cancer, bladder cancer, cervical cancer, lung cancer, breast cancer and skin cancer.

Inhibitors of the cyclooxygenase pathway in the metabolism of arachidonic acid used in the prevention and treatment of cancer or neoplasia may inhibit enzyme activity through a variety of mechanisms. The use of cyclooxygenasse-2 selective inhibitors is highly advantageous in that it minimize the gastric side effects that can occur with non-selective NSAID's, especially where prolonged prophylactic treatment is expected.

The term “cyclooxygenase-2 inhibitor” denotes a compound able to inhibit cyclooxygenase-2 without significant inhibition of cyclooxygenase-1. Preferably, it includes compounds which have a cyclooxygenase-2 IC₅₀ of less than about 0.2 μM, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 μM, and more preferably of greater than 10 μM.

The term “purified” means partially purified and/or completely purified. Thus a “purified composition” may be either partially purified or completely purified. An extract of a naturally occurring cyclooxygenase-2 inhibitor may be paritially purified or purified.

Uroguanylin is secreted naturally by the goblet cells of the intestinal mucosal lining as prouroguanylin, a functionally inactive form, which is then converted to the functionally active uroguanylin in the intestine by endogenous proteases. Uroguanylin is an acid-stable, proteolysis-resistant peptide. Therefore, orally delivered prouroguanylin and uroguanylin will act on the lumenal intestinal surface and not be absorbed systemically. Oral administration of uroguanylin, prouroguanylin and other like peptides, containing the amino acid sequences similar to the active domain, are expected to induce apoptosis, cell death, in the intestinal mucosal cell lining. The induced apoptosis in the intestinal mucosal cell lining is expected to retard the incidence of polyp formation and subsequent intestinal cancer. Without intending to be bound by any theory, applicants believe that the peptides of the invention exert their effects by increasing the rate of apoptosis, cell death, in the intestinal mucosal cell lining promoting the perfect balance between the cell proliferation and the programmed cell death thereby retarding the growth of polyps and preventing, inhibiting, and treating cancer in the intestine and other epithelial-derived cancer possessing receptors for guanylin, uroguanylin, lymphoguanylin and STa family of peptides.

The rate of cell proliferation and cell death in the intestinal mucosa is very rapid. The cells of the intestinal mucosa are in a steady state of turnover to insure a perfect balance between cell proliferation and cell death. The constant rapid renewal of the GI tract epithelium fulfills the functions of maintaining the integrity of normal mucosa, repairing and replenishing differentiated epithelial cells that have specialized functions. The prevention of apoptosis in the intestinal mucosal cells creating an imbalance in the renewal process results in an increased incidence of polyp formation and subsequent intestinal cancer. See Eastwood et al, A review of gastrointestinal epithelial renewal and its relevance to the development of adenocarcinomas of the gastrointestinal tract, J. Clin. Gastroenterol. 21: S1-11. (1995). The process of apoptosis is known to be suppressed in colon cancer tissues. Baretton, et al, Apoptosis and immunohistochemical bcl-2 expression in colorectal adenomoas and carcinomas. Aspectes of Carcinogenesis and prognostic significance, Cancer 77:255-264 (1996).

A major cellular characteristic of the apoptotic process is a marked loss of cell volume, which is directly related to the movement of ions, with homeostatsis being achieved by the balance of osmotic pressure across the plasma membrane. Hoffman, E. K. et al, Membrane mechanisms in intracellular signalling in cell volume regulation, Int. Rev. Cytol. 161:173-262 (1995). Most mammalian cells achieve and maintain this osmotic pressure through the continuous action of Na⁺/K⁺ ATPase pump, which creates a gradient of these monovalent cations across the membrane. Several sources of evidence have implicated a potential role of K⁺ efflux in the induction of apoptosis. Hughes, F. M. et al, Intracellular K⁺ suppresses the activation of apoptosis in lymphocytes, J. Biol. Chem. 272:30567-30576 (1997); Hughes, F. M. et al, Potassium is a critical regulator of apoptotic enzymes in vitro and in vivo, Adv. Enzyme Regul. 39:157-171 (1999). First, a bacterial pore-forming cytolysin, staphylococcal α-toxin, which selectively permeabilizes plasma membranes for monovalent cations, was found to induce apoptosis. Bhakdi, S. et al, Release of interleukin-1 beta associated with potent cytocidal action of staphylococcal alpha-toxin on human monocytes, Infect. Immun. 57:3512-3519 (1989). Second, apoptotic and shrunken cells have been shown to contain much lower levels of intracellular K⁺ as compared to that in normal cells. Hughes, F. M et al, Intracellular K+ suppresses the activation of apoptosis in lymphocytes, J. Biol. Chem. 272:30567-30576 (1997). Third, an intracellular K+ concentration more than 150 mM has been shown to selectively inhibit Caspase-3, a proteolytic enzyme involved in the induction of apoptosis. Hughes, F. M. et al, Potassium is a critical regulator of apoptotic enzymes in vitro and in vivo, Adv. Enzyme Regul. 39:157-171 (1999). Finally, suppressing K⁺ efflux in whole cells prevents the activation of pro-apoptosis nucleases, whereas enhancing the efflux of this ion facilitates enzymatic activities of these nucleases. Hughes, F. M. 39: 157-171 (1999). Thus, intracellular levels of potassium balance appear to be the critical regulator of apoptosis.

Additionally, guanylin has been shown to be completely diminished in colon cancer cells and evenly expressed in normal intestinal mucosal cells. This finding suggest that guanylin is involved in the maintenance of colonic differentiation or functions as a tumor modifier gene. Mitchell et al., Guanylin mRNA Expression in Human Intestine and Colorectal Adenocarcinoma, Lab. Invest. 1998, Vol. 78; No. 1, 101-108. Recent data demonstrates that the guanylin cyclase receptor known as GC-C receptor is expressed in all primary and metastatic colorectal cancers and it may serve as a specific marker for these tumors. Carrithers, S. L. et al, Guanylin cyclase C is a selective marker for metastatic colorectal tumors in human extraintestinal tissues, Proc. Natl. Acad. Sci. USA. 93:14827-14832. By contrast, the expression of guanylin has been shown to be down-regulated in colorectal cancer tissues and cell lines. Cohen, M. B. et al, Guanylin mRNA expression in human intestine and colorectal adenocarcinoma, Lab. Invest. 78:101-108.

In PCT/US00/21998 (herein incorporated by reference) uroguanylin was shown to be completely diminished in colon cancer cells and evenly distributed in normal intestinal mucosal cells. Additionally, the expression of uroguanylin and guanylin in human colon cancer and the adjacent normal tissues was reportedly completely diminished in all human colon cancer specimens examined. That study suggested that either the reduced expression of uroguanylin and/or guanylin leads to or is a result of adenocarcinoma formation. In the same application, it was demonstrated that treatment with uroguanylin resulted in the induction of apoptosis in T-84, human colon carcinoma cells, and that the oral administration of human uroguanylin leads to inhibition in polyp formation in the intestinal tract of Min-mouse, an animal model for human Familial Adenomatous Polyposis (FAP).

Both guanylin and uroguanylin genes have recently been mapped on the mouse chromosome 4 and to a synthetic position on human chromosome 1p34-35. Sciaky, D. et al, Mapping of guanylin to murine chromonsome 4 and human chromosome 1p34-35, Genomics 26:427-429 (1995); Whitaker, T. L. et al, The uroguanylin gene (Guca 1b) is linked to guanylin (Guca 2) on mouse chromosome 4, Genomics 45:348-354 (1997) This region is frequently associated with the loss of heterozygosity in human colon carcinoma. Leister, I. et al, Human colorectal cancer: high frequency of deletions at chromosome 1p35, Cancer Res. 50:7232-7235 (1990). In the min-mouse tumor model, ademona multiplicity and growth rate are regulated by APC, the tumor suppressor gene, which is also localized to mouse chromosome 4 in a region syntenic with human chromosome 1p34-36. Dietrich, W. F. et al, Genetic identification of Mom-1, a major modifier locus affecting Min-induced intestinal neoplasia in the mouse, Cell 75:631-639 (1992). The APC gene is mutated in the vast majority of humans with colorectal cancer. Miyoshi, Y. et al, Somatic mutations of the APC gene in colorectal tumors: mutation cluster region in the APC gene, Hum. Mol. Genet. 1:229-233 (1992). The principal function of this gene is to regulate cell cycle via the wnt signal transduction cascade. Cadigan, K. M. et al, Wnt signaling: a common theme in animal development, Genes Dev. 11:3286-3305 (1997). Thus, the uroguanylin and guanylin peptides may be involved early in the process of colon carcinogenesis.

In accordance with the process of the present invention, therefore, a polypeptide which contains the active domain of human uroguanylin or which binds to the guanylate cyclase receptor GC-C in the intestine of the subject is administered to a subject. While the polypeptide may be administered prophylactically, it will typically be administered to a subject who has been determined to have intestinal cancer, intestinal polyps, or a genetic predisposition for the growth of polyps in the intestine.

In a preferred embodiment of the present invention, the polypeptide is a polypeptide having the sequence as identified in SEQ. ID. 1:

X₈-Asp -Asp -Cys -X₁ -X₂ -Cys -X₃ -Asn -X₄ -X₅
-Cys -X₆ -X₇ -Cys-X₉

wherein each of X₁, X₂, X₃, X₄, X₅, X₆, and X₇ is an amino acid residue, X₈ and X₉ are independently hydrogen or at least one amino acid residue, and the polypeptide is cross-linked by a disulfide bond between the cystine residue immediately adjacent the amine group of X₁ and the cystine residue immediately adjacent the amine group of X₆ and by a disulfide bond between the cystine residue immediately adjacent the amine group of X₃ and the cystine residue immediately adjacent the carboxy group of X₇. Preferably, the polypeptide is guanylan, uroguanylin, pro-uroguanylin, or another polypeptide which contains the active domain of uroguanylin.

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within 2 are substituted for one another. More preferred substitutions are those wherein the amino acids have hydropathic indices within 1. Most preferred substitutions are those wherein the amino acids have hydropathic indices within 0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids, (according to the Hopp-Woods values): arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydropathic indices within 2 are preferably substituted for one another, those within +1 are more preferred, and those within ±0.5 are most preferred.

As outlined above, amino acid substitutions in the peptides of the present invention can be based on the relative similarity of the amino acid side-chain substituents in the non-active domain of the peptide to create a protein with the same biological activity as the human uroguanylin peptide. Thus, X₁ may be selected from the group of all amino acid residues, but preferably is selected from the group of amino acid residues consisting of aspartic acid, glutamic acid, glycine, lysine, asparagine, proline, glutamine, arginine, serine and threonine. The more preferred amino acid residues that may be substituted for X₁ are glutamic acid, aspartic acid, arginine, and lysine. The most preferred amino acid residue that may be used for X₁ is glutamic acid. X₂ may be selected from all amino acid residues, however the preferred amino acid residues for substitution are leucine, isoleucine, tyrosine, phenylalanine, tryptophan, valine, methionine, cysteine, alanine, histidine, proline, threonine, glycine, asparagine, and glutamine. The more preferred amino acid residues that may be substituted for X₂ are cysteine, phenylalanine, glycine, isoleucine, leucine, methionine, valine, and tyrosine. Among the more preferred amino acid residues mentioned above, the even more preferred amino acid residues for substitution for X₂ are leucine, isoleucine, tyrosine, valine, and methionine. The most preferred amino acid residue for substitution for X₂ is leucine.

Additionally, as discussed above, X₃ and X₄ may be selected from all amino acid residues, but the preferred amino acid residues are valine, isoleucine, tyrosine, phenylalanine, tryptophan, methionine, cysteine, alanine, histidine, proline, threonine, glycine, glutamine, asparagine, and serine. The more preferred amino acid residues that may be substituted for X₃ and X₄ are valine, isoleucine, leucine, tyrosine, phenylalanine, methionine, cysteine, alanine, histidine, and proline. Among the more preferred amino acid residues mentioned above, the even more preferred amino acid residues that may be substituted for X₃ and X₄ are valine, isoleucine, leucine, methionine, and cysteine. Even more preferable for substitution for X₃ and X₄ are isoleucine and valine. The most preferred amino acid residue for substitution for X₃ and X₄ is valine. Also, X₅ may be selected from all amino acid residues, but the preferred amino acid residues are alanine, histidine, cysteine, methionine, valine, leucine, isoleucine, tyrosine, phenylalanine, proline, threonine, glycine, glutamine, asparagine, and serine. The more preferred amino acid residues that may be substituted for X₅ are alanine, histidine, cysteine, methionine, valine, proline, threonine, glycine, glutamine, asparagine, and serine. Even more preferred amino acid residues for substitution for X₅ are alanine, histidine, cysteine, proline, threonine, glycine, glutamine, asparagine, and serine. The most preferred amino acid residue for substitution for X₅ is alanine.

Moreover, X₆ may be selected from all amino acid residues, but the preferred amino acid residues for substitution are threonine, proline, alanine, histidine, cysteine, methionine, valine, leucine, isoleucine, tyrosine, glycine, glutamine, asparagine, and serine. The more preferred amino acid residues for substitution for X₆ are threonine, proline, alanine, histidine, cysteine, methionine, glycine, glutamine, asparagine, and serine. Even more preferred amino acid residues for substitution threonine, proline, alanine, histidine, and glycine. The most preferred amino acid residue for substitution for X₆ is threonine. Also, X₇ may be selected from all amino acid residues, but the preferred amino acid residues are glycine, threonine, proline, alanine, histidine, cysteine, methionine, valine, leucine, isoleucine, glutamine, asparagine, serine, glutamic acid, and aspartic acid. The more preferred amino acid residues for substitution for X₇ are glycine, threonine, proline, alanine, histidine, cysteine, glutamine, asparagine, and serine. Even more preferred amino acid residues for substitution for X₇ are glycine, threonine, proline, alanine, histidine, glutamine, asparagine, and serine. The most preferred amino acid residue for substitution for X₇ is glycine.

The polypeptides and compounds of the present invention can be combined with various excipient vehicles and/or adjuvants well known in this art which serve as pharmaceutically acceptable carriers to permit drug administration in the form of, e.g., injections, suspensions, emulsions, tablets, capsules, and ointments. These pharmaceutical compositions may be administered by any acceptable means. For warm-blooded animals, and in particular, for humans, administration can be oral, parenteral, subcutaneous, intravenous, intramuscular and/or intraperitoneal. The specific dose administered will be dependent upon such factors as the general health and physical condition of the subject as well as the subject's age and weight, the stage of the subject's disease condition, the existence of any concurrent treatments, and the frequency of administration; typically, the dose will be in the range of about 0.5 to about 2.0 mg/kg for human subjects. In general, the composition will contain one or more of the polypeptide(s) of the present invention in a concentration of at least about 0.0001% by weight, more typically at least about 0.001% by weight, still more typically at least about 0.01%, still more typically at least about 0.1% and, in some embodiments, in a concentration of at least about 1% by weight of the composition.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

If the neoplasia is localized in the G.I. tract, the compound may be formulated with acid-stable, base-labile coatings known in the art which begin to dissolve in the high pH small intestine. Formulation to enhance local pharmacologic effects and reduce systemic uptake are preferred.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

Formulations for topical use include known gels, creams, oils, and the like. For aerosol delivery, the compounds may be formulated with known aerosol exipients, such as saline, and administered using commercially available nebulizers. Formulation in a fatty acid source may be used to enhance biocompatibility. Aerosol delivery is the preferred method of delivery for epithelial neoplasias of the lung for prevention application.

For rectal administration, the active ingredient may be formulated into suppositories using bases which are solid at room temperature and melt or dissolve at body temperature. Commonly used bases include coca butter, glycerinated gelatin, hydrogenated vegetable oil, polyethylene glycols of various molecular weights, and fatty esters of polyethylene stearate.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, the location of the neoplasia, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

Human uroguanylin cDNA has been cloned in bacteria, and chemically synthesized by solid phase peptide synthesis. Uroguanylin peptide can be chemically synthesized by using the procedure as described in U.S. Pat. No. 5,489,670 Human Uroguanylin and in U.S. Pat. No. 5,140,102 Pentadecapeptide, guanylin, which stimulates intestinal guanylate cyclase. Peptides similar to uroguanylin peptides have been identified in mouse, rat, porcine, and bovine species. The functionally active domain in most of these peptides are highly conserved. Therefore, the physiological functions of these peptides may be similar, and these peptides may be used as intestinal cancer preventative agents as well. Thus, as long as the functionally active domains of these peptides are conserved, substitutions in the non-active domains may be achieved with no change in the activity of the peptides.

In the present invention, the combination of any one or more of the following peptides; uroguanylin, human uroguanylin, pro-uroguanylin, and human pro-uroguanylin, guanylin, lymphoguanylin, prolymphoguanylin and heat stable enterotoxin, with any one of more of naturally occurring, or an extract of a natural occurring, or a chemically synthesized cyclooxygenase-2 inhibitor, preferably a selective cyclooxygenase-2 inhibitor or inhibitors is disclosed for the prevention, inhibition, or treatment of cancer in the intestinal tract by administration of an effective amount of such a combination to a subject in need of such treatment.

In such a combination, the cyclooxygenase inhibitor can be, by way of example, a COX-2 nonselective inhibitor or a COX-2 selective inhibitor. Examples of COX-2 nonselective inhibitors include the well-known compounds aspirin, acetaminophen, indomethacin, sulindac, etodolac, mefenamic acid, tolmetin, ketorolac, diclofenac, ibuprofen, naproxen, fenoprofen, ketoprofen, oxaprozin, flurbiprofen, piroxicam, tenoxicam, phenylbutazone, apazone, or nimesulide or a pharmaceutically acceptable salt or derivative or prodrug thereof. In a preferred embodiment of the invention the COX-2 nonselective inhibitor is selected from the group comprising aspirin, acetaminophen, indomethacin, ibuprofen, or naproxen.

In the preferred embodiments, the cyclooxygenase-2 inhibitor is selected from compounds of Formula I

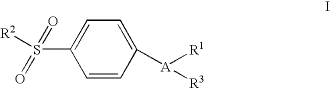

wherein A is a substituent selected from partially unsaturated or unsaturated heterocyclyl and partially unsaturated or unsaturated carbocyclic rings;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl or amino; and

wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically-acceptable salt thereof.

A preferred class of compounds which inhibit cyclooxygenase-2 consists of compounds of Formula I wherein A is selected from 5- or 6-member partially unsaturated heterocyclyl, 5- or 6-member unsaturated heterocyclyl, 9- or 10-member unsaturated condensed heterocyclyl, lower cycloalkenyl and phenyl; wherein R¹ is selected from 5- and 6-membered heterocyclyl, lower cycloalkyl, lower cycloalkenyl and aryl selected from phenyl, biphenyl and naphthyl, wherein R¹ is optionally substituted at a substitutable position with one or more radicals selected from lower alkyl, lower haloalkyl, cyano, carboxyl, lower alkoxycarbonyl, hydroxyl, lower hydroxyalkyl, lower haloalkoxy, amino, lower alkylamino, phenylamino, lower alkoxyalkyl, lower alkylsulfinyl, halo, lower alkoxy and lower alkylthio; wherein R² is methyl or amino; and wherein R³ is a radical selected from hydrido, oxo, cyano, carboxyl, lower alkoxycarbonyl, lower carboxyalkyl, lower cyanoalkyl, halo, lower alkyl, lower alkyloxy, lower cycloalkyl, phenyl, lower haloalkyl, 5- or 6-membered heterocyclyl, lower hydroxylalkyl, lower aralkyl, acyl, phenylcarbonyl, lower alkoxyalkyl, 5- or 6-membered heteroaryloxy, aminocarbonyl, lower alkylaminocarbonyl, lower alkylamino, lower aminoalkyl, lower alkylaminoalkyl, phenyloxy, and lower aralkoxy; or a pharmaceutically-acceptable salt thereof.

A more preferred class of compounds which inhibit cyclooxygenase-2 consists of compounds of Formula I wherein A is selected from oxazolyl, isoxazolyl, furyl, thienyl, dihydrofuryl, pyrrolyl, pyrazolyl, thiazolyl, imidazolyl, isothiazolyl, benzofuryl, cyclopentenyl, cyclopentadienyl, phenyl, and pyridyl; wherein R¹ is selected from pyridyl optionally substituted at a substitutable position with one or more methyl radicals, and phenyl optionally substituted at a substitutable position with one or more radicals selected from methyl, ethyl, isopropyl, butyl, tert-butyl, isobutyl, pentyl, hexyl, fluoromethyl, difluoromethyl, trifluoromethyl, cyano, carboxyl, methoxycarbonyl, ethoxycarbonyl, hydroxyl, hydroxymethyl, trifluoromethoxy, amino, N-methylamino, N,N-dimethylamino, N-ethylamino, N,N-dipropylamino, N-butylamino, N-methyl-N-ethylamino, phenylamino, methoxymethyl, methylsulfinyl, fluoro, chloro, bromo, methoxy, ethoxy, propoxy, n-butoxy, pentoxy, and methylthio; wherein R² is methyl or amino; and wherein R³ is a radical selected from hydrido, oxo, cyano, carboxyl, methoxycarbonyl, ethoxycarbonyl, carboxypropyl, carboxymethyl, carboxyethyl, cyanomethyl, fluoro, chloro, bromo, methyl, ethyl, isopropyl, butyl, tert-butyl, isobutyl, pentyl, hexyl, difluoromethyl, trifluoromethyl, pentafluoroethyl, heptafluoropropyl, difluoroethyl, difluoropropyl, methoxy, ethoxy, propoxy, n-butoxy, pentoxy, cyclohexyl, phenyl, pyridyl, thienyl, thiazolyl, oxazolyl, furyl, pyrazinyl, hydroxylmethyl, hydroxylpropyl, benzyl, formyl, phenylcarbonyl, methoxymethyl, furylmethyloxy, aminocarbonyl, N-methylaminocarbonyl, N,N-dimethylaminocarbonyl, N,N-dimethylamino, N-ethylamino, N,N-dipropylamino, N-butylamino, N-methyl-N-ethylamino, aminomethyl, N,N-dimethylaminomethyl, N-methyl-N-ethylaminomethyl, benzyloxy, and phenyloxy; or a pharmaceutically-acceptable salt thereof.

A family of specific compounds of particular interest within Formula I consists of compounds and pharmaceutically-acceptable salts thereof as follows:

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)pyrazole;

4-(5-(4-chlorophenyl)-3-(4-methoxyphenyl)-1H-pyrazol-1-yl)benzenesulfonamide

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide

4-[5-(4-chlorophenyl)-3-(trifluoromethyl)-1H-pyrazol-1-yl]benzenesulfonamide;

4-[5-phenyl-3-(trifluoromethyl)-1H-pyrazol-1-yl]benzenesulfonamide;

4-[5-(4-fluorophenyl)-3-(trifluoromethyl)-1H-pyrazol-1-yl]benzenesulfonamide;

4-[5-(4-methoxyphenyl)-3-(trifluoromethyl)-1H-pyrazol-1-yl]benzenesulfonamide;

4-[5-(4-chlorophenyl)-3-(difluoromethyl)-1H-pyrazol-1-yl]benzenesulfonamide;

4-[5-(4-methylphenyl)-3-(trifluoromethyl)-1H-pyrazol-1-yl]benzenesulfonamide;

4-[4-chloro-5-(4-chlorophenyl)-3-(trifluoromethyl)-1H-pyrazol-1-yl]benzenesulfonamide;

4-[3-(difluoromethyl)-5-(4-methylphenyl)-1H-pyrazol-1-yl]benzenesulfonamide;

4-[3-(difluoromethyl)-5-phenyl-1H-pyrazol-1-yl]benzenesulfonamide;

4-[3-(difluoromethyl)-5-(4-methoxyphenyl)-1H-pyrazol-1-yl]benzenesulfonamide;

4-[3-cyano-5-(4-fluorophenyl)-1H-pyrazol-1-yl]benzenesulfonamide;

4-[3-(difluoromethyl)-5-(3-fluoro-4-methoxyphenyl)-1H-pyrazol-1-yl]benzenesulfonamide;

4-[5-(3-fluoro-4-methoxyphenyl)-3-(trifluoromethyl)-1H-pyrazol-1-yl]benzenesulfonamide;

4-[4-chloro-5-phenyl-1H-pyrazol-1-yl]benzenesulfonamide;

4-[5-(4-chlorophenyl)-3-(hydroxymethyl)-1H-pyrazol-1-yl]benzenesulfonamide;

4-[5-(4-(N,N-dimethylamino)phenyl)-3-(trifluoromethyl)-1H-pyrazol-1-yl]benzenesulfonamide;

5-(4-fluorophenyl)-6-[4-(methylsulfonyl)phenyl]spiro[2.4]hept-5-ene;

4-[6-(4-fluorophenyl)spiro[2.4]hept-5-en-5-yl]benzenesulfonamide;

6-(4-fluorophenyl)-7-[4-(methylsulfonyl)phenyl]spiro[3.4]oct-6-ene;

5-(3-chloro-4-methoxyphenyl)-6-[4-(methylsulfonyl)phenyl]spiro[2.4]hept-5-ene;

4-[6-(3-chloro-4-methoxyphenyl)spiro[2.4]hept-5-en-5-yl]benzenesulfonamide;

5-(3,5-dichloro-4-methoxyphenyl)-6-[4-(methylsulfonyl)phenyl]spiro[2.4]hept-5-ene;

5-(3-chloro-4-fluorophenyl)-6-[4-(methylsulfonyl)phenyl]spiro[2.4]hept-5-ene;

4-[6-(3,4-dichlorophenyl)spiro[2.4]hept-5-en-5-yl]benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluoromethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzylaminothiazole;

4-(4-fluorophenyl)-5-(4-methylsulfonylphenyl)-2-(1-propylamino)thiazole;

2-[(3,5-dichlorophenoxy)methyl)-4-(4-fluorophenyl)-5-(4-(methylsulfonyl)phenyl]thiazole;

5-(4-fluorophenyl)-4-(4-methylsulfonylphenyl)-2-trifluoromethylthiazole;

1-methylsulfonyl-4-[1,1-dimethyl-4-(4-fluorophenyl)cyclopenta-2,4-dien-3-yl]benzene;

4-[4-(4-fluorophenyl)-1,1-dimethylcyclopenta-2,4-dien-3-yl]benzenesulfonamide;

5-(4-fluorophenyl)-6-[4-(methylsulfonyl)phenyl]spiro[2.4]hepta-4,6-diene;

4-[6-(4-fluorophenyl)spiro[2.4]hepta-4,6-dien-5-yl]benzenesulfonamide;

6-(4-fluorophenyl)-2-methoxy-5-[4-(methylsulfonyl)phenyl]-pyridine-3-carbonitrile;

2-bromo-6-(4-fluorophenyl)-5-[4-(methylsulfonyl)phenyl]-pyridine-3-carbonitrile;

6-(4-fluorophenyl)-5-[4-(methylsulfonyl)phenyl]-2-phenyl-pyridine-3-carbonitrile;

4-[2-(4-methylpyridin-2-yl)-4-(trifluoromethyl)-1H-imidazol-1-yl]benzenesulfonamide;

4-[2-(5-methylpyridin-3-yl)-4-(trifluoromethyl)-1H-imidazol-1-yl]benzenesulfonamide;

4-[2-(2-methylpyridin-3-yl)-4-(trifluoromethyl)-1H-imidazol-1-yl]benzenesulfonamide;

3-[1-[4-(methylsulfonyl)phenyl]-4-(trifluoromethyl)-1H-imidazol-2-yl]pyridine;

2-[1-(4-(methylsulfonyl)phenyl-4-(trifluoromethyl)-1H-imidazol-2-yl]pyridine;

2-methyl-4-[1-[4-(methylsulfonyl)phenyl-4-(trifluoromethyl)-1H-imidazol-2-yl]pyridine;

2-methyl-6-[1-[4-(methylsulfonyl)phenyl-4-(trifluoromethyl)-1H-imidazol-2-yl]pyridine;

4-[2-(6-methylpyridin-3-yl)-4-(trifluoromethyl)-1H-imidazol-1-yl]benzenesulfonamide;

2-(3,4-difluorophenyl)-1-[4-(methylsulfonyl)phenyl]-4-(trifluoromethyl)-1H-imidazole;

4-[2-(4-methylphenyl)-4-(trifluoromethyl)-1H-imidazol-1-yl]benzenesulfonamide;

2-(4-chlorophenyl)-1-[4-(methylsulfonyl)phenyl]-4-methyl-1H-imidazole;

2-(4-chlorophenyl)-1-[4-(methylsulfonyl)phenyl]-4-phenyl-1H-imidazole;

2-(4-chlorophenyl)-4-(4-fluorophenyl)-1-[4-(methylsulfonyl)phenyl]-1H-imidazole;

2-(3-fluoro-4-methoxyphenyl)-1-[4-(methylsulfonyl)phenyl-4-(trifluoromethyl)-1H-imidazole;

1-[4-(methylsulfonyl)phenyl]-2-phenyl-4-trifluoromethyl-1H-imidazole;

2-(4-methylphenyl)-1-[4-(methylsulfonyl)phenyl]-4-trifluoromethyl-1H-imidazole;

4-[2-(3-chloro-4-methylphenyl)-4-(trifluoromethyl)-1H-imidazol-1-yl]benzenesulfonamide;

2-(3-fluoro-5-methylphenyl)-1-[4-(methylsulfonyl)phenyl]-4-(trifluoromethyl)-1H-imidazole;

4-[2-(3-fluoro-5-methylphenyl)-4-(trifluoromethyl)-1H-imidazol-1-yl]benzenesulfonamide;

2-(3-methylphenyl)-1-[4-(methylsulfonyl)phenyl-3-4-trifluoromethyl-1H-imidazole;

4-[2-(3-methylphenyl)-4-trifluoromethyl-1H-imidazol-1-yl]benzenesulfonamide;

1-[4-(methylsulfonyl)phenyl]-2-(3-chlorophenyl)-4-trifluoromethyl-1H-imidazole;

4-[2-(3-chlorophenyl)-4-trifluoromethyl-1H-imidazol-1-yl]benzenesulfonamide;

4-[2-phenyl-4-trifluoromethyl-1H-imidazol-1-yl]benzenesulfonamide;

4-[2-(4-methoxy-3-chlorophenyl)-4-trifluoromethyl-1H-imidazol-1-yl)benzenesulfonamide;

1-allyl-4-(4-fluorophenyl)-3-[4-(methylsulfonyl)phenyl]-5-(trifluoromethyl)-1H-pyrazole;

4-[1-ethyl-4-(4-fluorophenyl)-5-(trifluoromethyl)-1H-pyrazol-3-yl]benzenesulfonamide;

N-phenyl-[4-(4-luorophenyl)-3-[4-(methylsulfonyl)phenyl]-5-(trifluoromethyl)-1H-pyrazol-1-yl]acetamide;

ethyl [4-(4-fluorophenyl)-3-[4-(methylsulfonyl)phenyl]-5-(trifluoromethyl)-1H-pyrazol-1-yl]acetate;

4-(4-fluorophenyl)-3-[4-(methylsulfonyl)phenyl]-1-(2-phenylethyl)-1H-pyrazole;

4-(4-fluorophenyl)-3-[4-(methylsulfonyl)phenyl]-1-(2-phenylethyl)-5-(trifluoromethyl)pyrazole;

1-ethyl-4-(4-fluorophenyl)-3-[4-(methylsulfonyl)phenyl]-5-(trifluoromethyl)-1H-pyrazole;

5-(4-fluorophenyl)-4-(4-methylsulfonylphenyl)-2-trifluoromethyl-1H-imidazole;

4-[4-(methylsulfonyl)phenyl]-5-(2-thiophenyl)-2-(trifluoromethyl)-1H-imidazole;

5-(4-fluorophenyl)-2-methoxy-4-(4-(methylsulfonyl)phenyl]-6-(trifluoromethyl)pyridine;

2-ethoxy-5-(4-fluorophenyl)-4-[4-(methylsulfonyl)phenyl]-6-(trifluoromethyl)pyridine;

5-(4-fluorophenyl)-4-[4-(methylsulfonyl)phenyl]-2-(2-propynyloxy)-6-(trifluoromethyl)pyridine;

2-bromo-5-(4-fluorophenyl)-4-[4-(methylsulfonyl)phenyl]-6-(trifluoromethyl)pyridine;

4-[2-(3-chloro-4-methoxyphenyl)-4,5-difluorophenyl]benzenesulfonamide;

1-(4-fluorophenyl)-2-[4-(methylsulfonyl)phenyl]benzene;

5-difluoromethyl-4-(4-methylsulfonylphenyl)-3-phenylisoxazole;

4-[(3-ethyl-5-phenylisoxazol-4-yl]benzenesulfonamide;

4-[5-difluoromethyl-3-phenylisoxazol-4-yl]benzenesulfonamide;

4-[5-hydroxymethyl-3-phenylisoxazol-4-yl]benzenesulfonamide;

4-[5-methyl-3-phenyl-isoxazol-4-yl]benzenesulfonamide;

1-[2-(4-fluorophenyl)cyclopenten-1-yl]-4-(methylsulfonyl)benzene;

1-[2-(4-fluoro-2-methylphenyl)cyclopenten-1-yl]-4-(methylsulfonyl)benzene;

1-[2-(4-chlorophenyl)cyclopenten-1-yl]-4-(methylsulfonyl)benzene;

1-[2-(2,4-dichlorophenyl)cyclopenten-1-yl)-4-(methylsulfonyl)benzene;

1-[2-(4-trifluoromethylphenyl)cyclopenten-1-yl]-4-(methylsulfonyl)benzene;

b 1-[2-(4-methylthiophenyl) cyclopenten-1-yl]-4-(methylsulfonyl)benzene;

1-[2-(4-fluorophenyl)-4,4-dimethylcyclopenten-1-yl]-4-(methylsulfonyl)benzene;

4-[2-(4-fluorophenyl)-4,4-dimethylcyclopenten-1-yl]benzenesulfonamide;

1-[2-(4-chlorophenyl)-4,4-dimethylcyclopenten-1-yl]-4-(methylsulfonyl)benzene;

4-[2-(4-chlorophenyl)-4,4-dimethylcyclopenten-1-yl]benzenesulfonamide;

4-[2-(4-fluorophenyl)cyclopenten-1-yl]benzenesulfonamide;

4-[2-(4-chlorophenyl)cyclopenten-1-yl]benzenesulfonamide;

1-[2-(4-methoxyphenyl)cyclopenten-1-yl]-4-(methylsulfonyl)benzene;

1-[2-(2,3-difluorophenyl)cyclopenten-1-yl]-4-(methylsulfonyl)benzene;

4-[2-(3-fluoro-4-methoxyphenyl)cyclopenten-1-yl]benzenesulfonamide;

1-[2-(3-chloro-4-methoxyphenyl)cyclopenten-1-yl]-4-(methylsulfonyl)benzene;

4-[2-(3-chloro-4-fluorophenyl)cyclopenten-1-yl]benzenesulfonamide;

4-[2-(2-methylpyridin-5-yl)cyclopenten-1-yl]benzenesulfonamide;

ethyl 2-[4-(4-fluorophenyl)-5-[4-(methylsulfonyl) phenyl]oxazol-2-yl]-2-benzyl-acetate;

2-[4-(4-fluorophenyl)-5-[4-(methylsulfonyl)phenyl]oxazol-2-yl]acetic acid;

2-(tert-butyl)-4-(4-fluorophenyl)-5-[4-(methylsulfonyl)phenyl]oxazole;

4-(4-fluorophenyl)-5-[4-(methylsulfonyl)phenyl]-2-phenyloxazole;

4-(4-fluorophenyl)-2-methyl-5-[4-(methylsulfonyl)phenyl]oxazole; and

4-[5-(3-fluoro-4-methoxyphenyl)-2-trifluoromethyl-4-oxazolyl]benzenesulfonamide.

A family of specific compounds of more particular interest within Formula I consists of compounds and pharmaceutically-acceptable salts thereof as follows:

4-[5-(4-chlorophenyl)-3-(trifluoromethyl)-1H-pyrazol-1-yl]benzenesulfonamide;

4-[5-(4-methylphenyl)-3-(trifluoromethyl)-1H-pyrazol-1-yl]benzenesulfonamide;

4-[5-(3-fluoro-4-methoxyphenyl)-3-(difluoromethyl)-1H-pyrazol-1-yl]benzenesulfonamide;

3-[1-[4-(methylsulfonyl)phenyl]-4-trifluoromethyl-1H-imidazol-2-yl]pyridine;

2-methyl-5-[1-[4-(methylsulfonyl)phenyl]-4-trifluoromethyl-1H-imidazol-2-yl]pyridine;

4-[2-(5-methylpyridin-3-yl)-4-(trifluoromethyl)-1H-imidazol-1-yl]benzenesulfonamide;

4-[5-methyl-3-phenylisoxazol-4-yl]benzenesulfonamide;

4-[5-hydroxymethyl-3-phenylisoxazol-4-yl]benzenesulfonamide;

[2-trifluoromethyl-5-(3,4-difluorophenyl)-4-oxazolyl]benzenesulfonamide;

4-[2-methyl-4-phenyl-5-oxazolyl]benzenesulfonamide; and

4-[5-(3-fluoro-4-methoxyphenyl-2-trifluoromethyl)-4-oxazolyl]benzenesulfonamide.

A subclass of cyclooxygenase-2 inhibitors is selected from compounds of Formula II

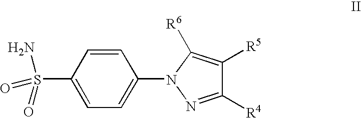

wherein R⁴ is selected from hydrido, alkyl, haloalkyl, alkoxycarbonyl, cyano, cyanoalkyl, carboxyl, aminocarbonyl, alkylaminocarbonyl, cycloalkylaminocarbonyl, arylaminocarbonyl, carboxyalkylaminocarbonyl, carboxyalkyl, aralkoxycarbonylalkylaminocarbonyl, aminocarbonylalkyl, alkoxycarbonylcyanoalkenyl and hydroxyalkyl;

wherein R⁵ is selected from hydrido, alkyl, cyano, hydroxyalkyl, cycloalkyl, alkylsulfonyl and halo; and

wherein R⁶ is selected from aralkenyl, aryl, cycloalkyl, cycloalkenyl and heterocyclic; wherein R⁴ is optionally substituted at a substitutable position with one or more radicals selected from halo, alkylthio, alkylsulfonyl, cyano, nitro, haloalkyl, alkyl, hydroxyl, alkenyl, hydroxyalkyl, carboxyl, cycloalkyl, alkylamino, dialkylamino, alkoxycarbonyl, aminocarbonyl, alkoxy, haloalkoxy, sulfamyl, heterocyclic and amino;

or a pharmaceutically-acceptable salt or derivative thereof.

A class of compounds of particular interest consists of those compounds of Formula I wherein R⁴ is selected from hydrido, lower alkyl, lower haloalkyl, lower alkoxycarbonyl, cyano, lower cyanoalkyl, carboxyl, aminocarbonyl, lower alkylaminocarbonyl, lower cycloalkylaminocarbonyl, arylaminocarbonyl, lower carboxyalkylaminocarbonyl, lower aminocarbonylalkyl, lower aralkoxycarbonylalkylaminocarbonyl, lower carboxyalkyl, lower alkoxycarbonylcyanoalkenyl and lower hydroxyalkyl; wherein R⁵ is selected from hydrido, lower alkyl, cyano, lower hydroxyalkyl, lower cycloalkyl, lower alkylsulfonyl and halo; and wherein R⁶ is selected from aralkenyl, aryl, cycloalkyl, cycloalkenyl and heterocyclic; wherein R⁴ is optionally substituted at a substitutable position with one or more radicals selected from halo, lower alkylthio, lower alkylsulfonyl, cyano, nitro, lower haloalkyl, lower alkyl, hydroxyl, lower alkenyl, lower hydroxyalkyl, carboxyl, lower cycloalkyl, lower alkylamino, lower dialkylamino, lower alkoxycarbonyl, aminocarbonyl, lower alkoxy, lower haloalkoxy, sulfamyl, five or six membered heterocyclic and amino; or a pharmaceutically-acceptable salt or derivative thereof.

A family of specific compounds of particular interest within Formula I consists of compounds, derivatives and pharmaceutically-acceptable salts thereof as follows:

4-[5-(4-chlorophenyl)-3-(trifluoromethyl)-1H-pyrazol-1-yl]benzenesulfonamide;

4-[5-phenyl-3-(trifluoromethyl)-1H-pyrazol-1-yl]benzenesulfonamide;

4-[5-(4-fluorophenyl)-3-(trifluoromethyl)-1H-pyrazol-1-yl]benzenesulfonamide;

4-[5-(4-methoxyphenyl)-3-(trifluoromethyl)-1H-pyrazol-1-yl]benzenesulfonamide;

4-[5-(4-chlorophenyl)-3-(difluoromethyl)-1H-pyrazol-1-yl]benzenesulfonamide;

4-[5-(4-methylphenyl)-3-(trifluoromethyl)-1H-pyrazol-1-yl]benzenesulfonamide;

4-[4-chloro-5-(4-chlorophenyl)-3-(trifluoromethyl)-1H-pyrazol-1-yl)benzenesulfonamide;

4-[3-(difluoromethyl)-5-(4-methylphenyl)-1H-pyrazol-1-yl]benzenesulfonamide;

4-[3-(difluoromethyl)-5-phenyl-1H-pyrazol-1-yl]benzenesulfonamide;

4-[3-(difluoromethyl)-5-(4-methoxyphenyl)-1H-pyrazol-1-yl]benzenesulfonamide;

4-[3-cyano-5-(4-fluorophenyl)-1H-pyrazol-1-yl]benzenesulfonamide;

4-[3-(difluoromethyl)-5-(3-fluoro-4-methoxyphenyl)-1H-pyrazol-1-yl]benzenesulfonamide;

4-[5-(3-fluoro-4-methoxyphenyl)-3-(trifluoromethyl)-1H-pyrazol-1-yl]benzenesulfonamide;

4-[4-chloro-5-phenyl-1H-pyrazol-1-yl]benzenesulfonamide;

4-[5-(4-chlorophenyl)-3-(hydroxymethyl)-1H-pyrazol-1-yl]benzenesulfonamide; and

4-[5-(4-(N,N-dimethylamino)phenyl)-3-(trifluoromethyl)-1H-pyrazol-1-yl]benzenesulfonamide.

A family of specific compounds of more particular interest within Formula I consists of compounds and pharmaceutically-acceptable salts or derivatives thereof as follows:

4-[5-(4-methylphenyl)-3-(trifluoromethyl)-1H-pyrazol-1-yl]benzenesulfonamide;

4-[5-(4-chlorophenyl)-3-(difluoromethyl)-1H-pyrazol-1-yl]benzenesulfonamide; and

4-[5-(3-fluoro-4-methoxyphenyl)-3-(difluoromethyl)-1H-pyrazol-1-yl]benzenesulfonamide.

Derivatives are intended to encompass any compounds which are structurally related to the cyclooxygenase-2 inhibitors or which possess the substantially equivalent biologic activity. By way of example, such inhibitors may include, but are not limited to, prodrugs thereof.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl. The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like. The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, ]5 cyclopentenyl, cyclopentadienyl, and cyclohexenyl. The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy. The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl. The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino. The term “alkylthio” embraces radicals containing a linear or branched alkyl radicals of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl. The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl. The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH₂O₂S—. The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl. The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted. The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl. The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl. The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable. The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy. The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical. The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups which have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups which have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl. The term “aminocarbonyl” denotes an amide group of the formula —C(═O)NH₂. The term “alkylaminocarbonyl” denotes an aminocarbonyl group which has been Formula B-2 (CAS registry number 179382-91-3) or a pharmaceutically acceptable salt or derivative or prodrug thereof.

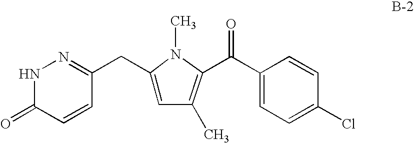

In a preferred embodiment of the invention the cyclooxygenase-2 selective inhibitor is a COX-2 selective inhibitor of the chromene structural class that is a substituted benzopyran or a substituted benzopyran analog selected from the group consisting of substituted benzothiopyrans, dihydroquinolines, or dihydronaphthalenes having the general Formula II shown below and possessing, by way of example and not limitation, the structures disclosed in Table 1, including the diastereomers, enantiomers, racemates, tautomers, salts, esters, amides and prodrugs thereof.

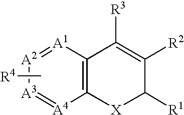

substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above. The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

The compounds utilized in the methods of the present invention may be present in the form of free bases or pharmaceutically acceptable acid addition salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, D-3-hydroxybutyric, salicylic, galactaric and galacturonic acid. Suitable pharmaceutically-acceptable base addition salts of compounds of Formula I include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound of Formula I by reacting, for example, the appropriate acid or base with the compound of Formula I.

In another embodiment of the invention the cyclooxygenase inhibitor can be a cyclooxygenase-2 selective inhibitor, for example, the COX-2 selective inhibitor meloxicam, Formula B-1 (CAS registry number 71125-38-7) or a pharmaceutically acceptable salt or derivative or prodrug thereof.

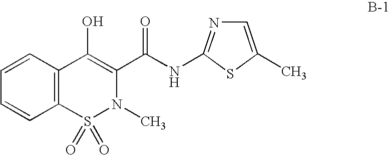

In yet another embodiment of the invention the cyclooxygenase-2 selective inhibitor is the COX-2 selective inhibitor RS 57067, 6-([5-(4-chlorobenzoyl)-1,4-dimethyl-1H-pyrrol-2-yl]methyl]-3(2H)-pyridazinone,

TABLE 1
Examples of Chromene COX-2 Selective
Inhibitors as Embodiments

| Compound | |
|---|---|
| Number | Structural Formula |
| B-3 | 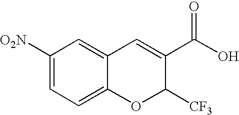 |
| | 6-Nitro-2-trifluoromethyl-2H-1- |
| | benzopyran-3-carboxylic acid |
| B-4 | 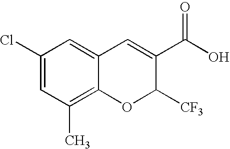 |
| | 6-Chloro-8-methyl-2-trifluoromethyl- |
| | 2H-1-benzopyran-3-carboxylic acid |
| B-5 | 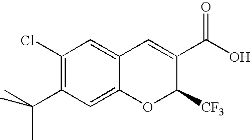 |
| | ((S)-6-Chloro-7-(1,1-dimethylethyl)-2-(trifluoromethyl- |
| | 2H-1-benzopyran-3-carboxylic acid |
| B-6 | 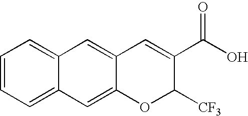 |
| | 2-Trifluoromethyl-2H-naphtho[2,3-b] |
| | pyran-3-carboxylic acid |
| B-7 | 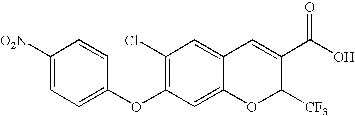 |
| | 6-Chloro-7-(4-nitrophenoxy)-2-(trifluoromethyl)-2H-1- |
| | benzopyran-3-carboxylic acid |
| B-8 | 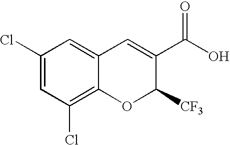 |
| | ((S)-6,8-Dichloro-2-(trifluoromethyl)- |
| | 2H-1-benzopyran-3-carboxylic acid |
| B-9 | 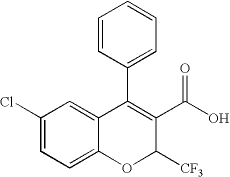 |
| | 6-Chloro-2-(trifluoromethyl)-4-phenyl-2H- |
| | 1-benzopyran-3-carboxylic acid |
| B-10 | 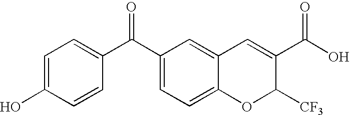 |
| | 6-(4-Hydroxybenzoyl)-2-(trifluoromethyl)- |
| | 2H-1-benzopyran-3-carboxylic acid |
| B-11 | 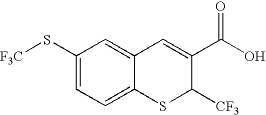 |
| | 2-(Trifluoromethyl)-6-[(trifluoromethyl)thio]- |
| | 2H-1-benzothiopyran-3-carboxylic acid |
| B-12 | 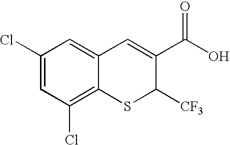 |
| | 6,8-Dichloro-2-trifluoromethyl-2H-1- |
| | benzothiopyran-3-carboxylic acid |
| B-13 | 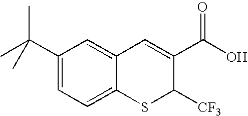 |
| | 6-(1,1-Dimethylethyl)-2-(trifluoromethyl)- |
| | 2H-1-benzothiopyran-3-carboxylic acid |
| B-14 | 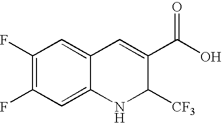 |
| | 6,7-Difluoro-1,2-dihydro-2-(trifluoromethyl)- |
| | 3-quinolinecarboxylic acid |
| B-15 | 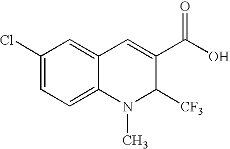 |
| | 6-Chloro-1,2-dihydro-1-methyl-2-(trifluoromethyl)- |
| | 3-quinolinecarboxylic acid |
| B-16 | 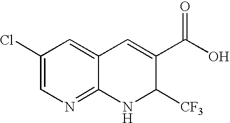 |
| | 6-Chloro-2-(trifluoromethyl)-1,2-dihydro |
| | [1,8]naphthyridine-3-carboxylic acid |
| B-17 | 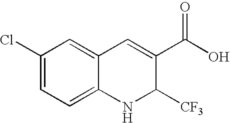 |
| | ((S)-6-Chloro-1,2-dihydro-2-(trifluoromethyl)- |
| | 3-quinolinecarboxylic acid |

In a more preferred embodiment of the invention the cycloxygenase-2 selective inhibitor is the substituted benzopyran (S)-6,8-dichloro-2-(trifluoromethyl)-2H-1-benzopyran-3-carboxylic acid, Formula B-8, or a pharmaceutically acceptable salt or derivative or prodrug thereof.

In a further preferred embodiment of the invention the cyclooxygenase inhibitor is selected from the class of tricyclic cyclooxygenase-2 selective inhibitors represented by the general structure of Formula III

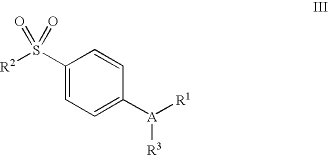

wherein A is a substituent selected from partially unsaturated or unsaturated heterocyclyl and partially unsaturated or unsaturated carbocyclic rings;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl or amino; and

wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt or derivative thereof.

In a still more preferred embodiment of the invention the cyclooxygenase-2 selective inhibitor represented by the above Formula III is selected from the group of compounds, illustrated in Table 2, consisting of celecoxib (B-18), valdecoxib (B-19), deracoxib (B-20), rofecoxib (B-21), etoricoxib (MK-663; B-22), JTE-522 (B-23), or a pharmaceutically acceptable salt or derivative or prodrug thereof.

TABLE 2
Examples of Tricyclic COX-2 Selective
Inhibitors as Embodiments

| Compound | |
|---|---|
| Number | Structural Formula |
| B-18 | 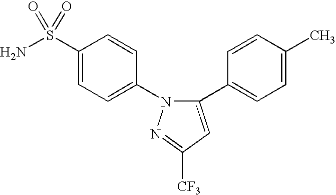 |
| B-19 | 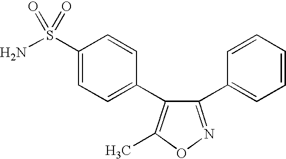 |
| B-20 | 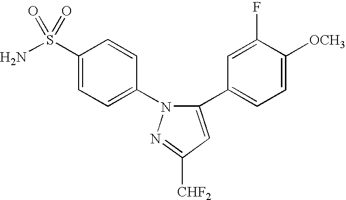 |
| B-21 | 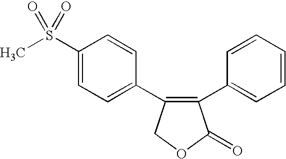 |
| B-22 | 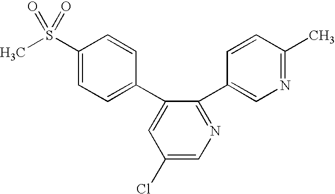 |
| B-23 | 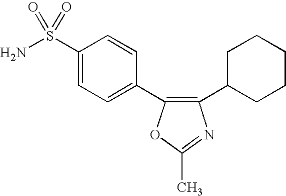 |

In an even more preferred embodiment of the invention the COX-2 selective inhibitor is selected from the group consisting of celecoxib, rofecoxib and etoricoxib.

In another highly preferred embodiment of the invention parecoxib, B-24, which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

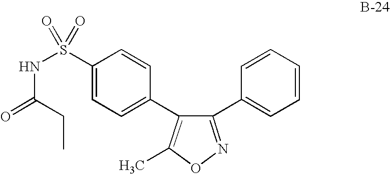

In another preferred embodiment of the invention, the compound ABT-963 having the formula B-25 that has been previously described in International Publication number WO 00/24719, is another tricyclic cyclooxygenase-2 selective inhibitor which may be advantageously employed.

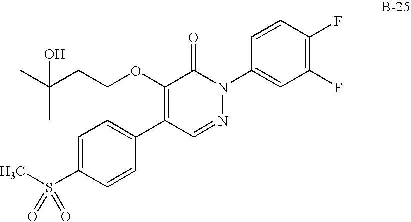

In a further preferred embodiment of the invention the cyclooxygenase inhibitor can be selected from the class of phenylacetic acid derivative cyclooxygenase-2 selective inhibitors represented by the general structure of Formula V:

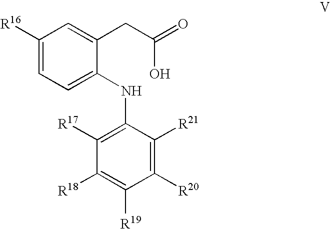

wherein R¹⁶ is methyl or ethyl;

R¹⁷ is chloro or fluoro;

R¹⁸ is hydrogen or fluoro

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor that is described in WO 99/11605 is a compound that has the designation of COX189 (CAS RN 346670-74-4), and that has the structure shown in Formula V,

wherein R¹⁶ is ethyl;

R¹⁷ and R¹⁹ are chloro;

R¹⁸ and R²⁰ are hydrogen; and

and R²¹ is methyl.

Other preferred cyclooxygenase-2 selective inhibitors that can be used in the present invention have the general structure shown in formula VI, where the J group is a carbocycle or a heterocycle. Particularly preferred embodiments have the structure:

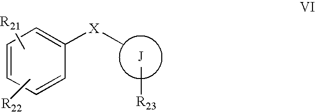

where:

X is O; J is 1-phenyl; R₂₁ is 2-NHSO₂CH₃; R₂₂ is 4-NO₂; and there is no R₂₃ group, (nimesulide), and

X is O; J is 1-oxo-inden-5-yl; R₂₁ is 2-F; R₂₂ is 4-F; and R₂₃ is 6-NHSO₂CH₃, (flosulide); and

X is O; J is cyclohexyl; R₂₁ is 2-NHSO₂CH₃; R₂₂ is 5-NO₂; and there is no R₂₃ group, (NS-398); and

X is S; J is 1-oxo-inden-5-yl; R₂₁ is 2-F; R₂₂ is 4-F; and R₂₃ is 6-N⁻SO₂CH₃.Na⁺, (L-745337); and

X is S; J is thiophen-2-yl; R₂₁ is 4-F; there is no R₂₂ group; and R₂₃ is 5-NHSO₂CH₃, (RWJ-63556); and

X is O; J is 2-oxo-5(R)-methyl-5-(2,2,2-trifluoroethyl)furan-(5H)-3-yl; R₂₁ is 3-F; R₂₂ is 4-F; and R₂₃ is 4-(p-SO₂CH₃)C₆H₄, (L-784512).

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in

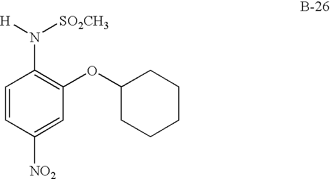

Japanese J. Cancer Res., 90(4):406-412 (1999); Falgueyret, J.-P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

An evaluation of the antiinflammatory activity of the cyclooxygenase-2 selective inhibitor, RWJ 63556, in a canine model of inflammation, was described by Kirchner et al., in J Pharmacol Exp Ther 282, 1094-1101 (1997).

Other compounds useful as the cyclooxygenase-2 selective inhibitor in the present invention include diarylmethylidenefuran derivatives such as those described in U.S. Pat. No. 6,180,651. Such diarylmethylidenefuran derivatives have the general formula shown below in formula VII:

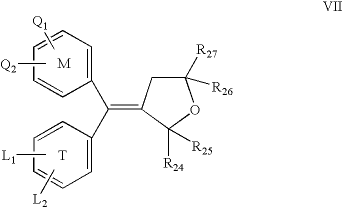

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q₁, Q₂, L₁ or L₂ is:

an —S(O)ₙ—R group, in which n is an integer equal to 0, 1 or 2 and R is a lower alkyl radical having 1 to 6 carbon atoms or a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q₁ and Q₂ or L₁ and L₂ are a methylenedioxy group; and

R₂₄, R₂₅, R₂₆ and R₂₇ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R₂₄, R₂₅ or R₂₆, R₂₇ are an oxygen atom, or

R₂₄, R₂₅ or R₂₆, R₂₇, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Preferred cyclooxygenase-2 selective inhibitors that are useful in the present invention include the following individual compounds; darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Another preferred embodiment of the invention, is the compound BMS-347070, having the formula:

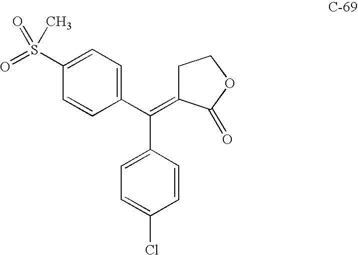

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

All references, patents or applications U.S. or foreign, cited in the application are hereby incorporated by reference as if written herein.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for the treatment or prevention of intestinal polyps in a subject, the method comprising administering to the subject an amount of a polypeptide and an amount of a cyclooxygenase-2 selective inhibitor wherein the amount of the polypeptide and the amount of the cyclooxygenase-2 selective inhibitor together comprise an intestinal polyp treating-effective amount of the polypeptide and the cyclooxygenase-2 selective inhibitor, wherein the polypeptide has the formula:
X₈-Asp -Asp -Cys -X₁ -X₂ -Cys -X₃ -Asn -X₄ -X₅
-Cys -X₆ -X₇ -Cys-X₉
and wherein each of X₁, X₂, X₃, X₄, X₅, X₆, and X₇ is an amino acid residue, X₈ and X₉ are independently hydrogen or at least one amino acid residue, and
the polypeptide is cross-linked by a disulfide bond between the cystine residue immediately adjacent the amine group of X₁ and the cystine residue immediately adjacent the amine group of X₆ and by a disulfide bond between the cystine residue immediately adjacent the amine group of X₃ and the cystine residue immediately adjacent the carboxy group of X₇.

2. A process of claim 1 wherein the polypeptide and cyclooxygenase-2 inhibitor are present as a single composition.

3. A process of claim 2 wherein the concentration of the peptide in the composition is at least 0.0001 percent by weight of the composition.

4. A process of claim 2 wherein the concentration of the peptide in the composition is at least 0.001 percent by weight of the composition.

5. A process of claim 2 wherein the concentration of the peptide in the composition is at least 0.01 percent by weight of the composition.

6. A process of claim 2 wherein the concentration of the peptide in the composition is at least 0.1 percent by weight of the composition.

7. A process of claim 2 wherein the concentration of the peptide in the composition is at least 1 percent by weight of the composition.

8. The process of claim 1 wherein said subject has been determined to have a genetic predisposition for the growth of polyps in the intestine.

9. The process of claim 1 wherein polyps have been identified in the intestine of said subject.

10. The process of claim 1 wherein said subject has been identified as having intestine cancer.

11. A process of claim 2 wherein the concentration of the peptide in the composition is at least 0.0001 percent by weight of the composition.

12. A process of claim 2 wherein the concentration of the peptide in the composition is at least 0.001 percent by weight of the composition.

13. A process of claim 2 wherein the concentration of the peptide in the composition is at least 0.01 percent by weight of the composition.

14. A process of claim 2 wherein the concentration of the peptide in the composition is at least 0.1 percent by weight of the composition.

15. A process of claim 2 wherein the concentration of the peptide in the composition is at least 1 percent by weight of the composition.

16. The process of claim 2 wherein said subject has been determined to have a genetic predisposition for the growth of polyps in the intestine.

17. The process of claim 2 wherein the polyps have been identified in the intestine of said subject.

18. The process of claim 2 wherein said subject has been identified as having intestine cancer.

19. The process of claim 1 wherein X₁ is selected from the group of amino acid residues consisting of aspartic acid, glutamic acid, glycine, lysine, asparagine, proline, glutamine, arginine, serine, and threonine.

20. The process of claim 1 wherein X₁ is selected from the group of amino acid residues consisting of glutamic acid, arginine, lysine, serine, aspartic acid, asparagine, glutamine, and glycine.

21. The process of claim 1 wherein X₁ is selected from the group of amino acid residues consisting of glutamic acid, aspartic acid, arginine, and lysine.

22. The process of claim 1 wherein X₁ is glutamic acid.

23. The process of claim 1 wherein X₂ is selected from the group of amino acid residues consisting of leucine, isoleucine, tyrosine, phenylalanine, tryptophan, valine, methionine, cysteine, alanine, histidine, proline, threonine, glycine, asparagine, and glutamine.

24. The process of claim 1 wherein X₂ is selected from the group of amino acid residues consisting of cysteine, phenylalanine, glycine, isoleucine, leucine, methionine, valine, and tyrosine.

25. The process of claim 1 wherein X₂ is selected from the group of amino acid residues consisting of leucine, isoleucine, tyrosine, valine, methionine.

26. The process of claim 1 wherein X₂ is selected from the group of amino acid residues consisting of leucine, and isoleucine.

27. The process of claim 1 wherein X₂ is leucine.

28. The process of claim 1 wherein X₃ is selected from the group of amino acid residues consisting of valine, isoleucine, leucine, tyrosine, phenylalanine, tryptophan, methionine, cysteine, alanine, histidine, proline, threonine, glycine, glutamine, asparagine, and serine.

29. The process of claim 1 wherein X₃ is selected from the group of amino acid residues consisting of valine, isoleucine, leucine, tyrosine, phenylalanine, methionine, cysteine, alanine, histidine, and proline.

30. The process of claim 1 wherein X₃ is selected from the group of amino acid residues consisting of valine, isoleucine, leucine, methionine, and cysteine.

31. The process of claim 1 wherein X₃ is valine.

32. The process of claim 1 wherein X₃ is isoleucine.

33. The process of claim 1 wherein X₄ is selected from the group of amino acid residues consisting of valine, isoleucine, leucine, tyrosine, phenylalanine, tryptophan, methionine, cysteine, alanine, histidine, proline, threonine, glycine, glutamine, asparagine, and serine.

34. The process of claim 1 wherein X₄ is selected from the group of amino acid residues consisting of valine, isoleucine, leucine, tyrosine, phenylalanine, methionine, cysteine, alanine, histidine, and proline.

35. The process of claim 1 wherein X₄ is selected from the group of amino acid residues consisting of valine, isoleucine, leucine, methionine, and cysteine.

36. The process of claim 1 wherein X₄ is valine.

37. The process of claim 1 wherein X₅ is alanine, histidine, cysteine, methionine, valine, leucine, isoleucine, tyrosine, phenylalanine, proline, threonine, glycine, glutamine, asparagine, and serine.

38. The process of claim 1 wherein X₅ is selected from the group of amino acid residues consisting of alanine, histidine, cysteine, methionine, valine, proline, threonine, glycine, glutamine, asparagine, and serine.

39. The process of claim 1 wherein X₅ is selected from the group of amino acid residues consisting of alanine, histidine, cysteine, proline, threonine, glycine, glutamine, asparagine, and serine.

40. The process of claim 1 wherein X₅ is alanine.

41. The process of claim 1 wherein X₆ is selected from the group of amino acid residues consisting of threonine, proline, alanine, histidine, cysteine, methionine, valine, leucine, isoleucine, tyrosine, glycine, glutamine, asparagine, and serine.

42. The process of claim 1 wherein X₆ is selected from the group of amino acid residues consisting of threonine, proline, alanine, histidine, cysteine, methionine, glycine, glutamine, asparagine, and serine.

43. The process of claim 1 wherein X₆ is selected from the group of amino acid residues consisting of threonine, proline, alanine, histidine, and glycine.

44. The process of claim 1 wherein X₆ is threonine.

45. The process of claim 1 wherein X₇ is selected from the group of amino acid residues consisting of glycine, threonine, proline, alanine, histidine, cysteine, methionine, valine, leucine, isoleucine, glutamine, asparagine, serine, glutamic acid, and aspartic acid.

46. The process of claim 1 wherein X₇ is selected from the group of amino acid residues consisting of glycine, threonine, proline, alanine, histidine, cysteine, glutamine, asparagine, and serine.

47. The process of claim 1 wherein X₇ is selected from the group of amino acid residues consisting of glycine, threonine, proline, alanine, histidine, glutamine, asparagine, and serine.

48. The process of claim 1 wherein X₇ is glycine.

49. The process of claim 1 wherein the polypeptide is uroguanylin.

50. The process of claim 1 wherein the polypeptide is human uroguanylin.

51. The process of claim 1 wherein the composition comprises pro-uroguanylin.

52. The process of claim 1 wherein the composition comprises human pro-uroguanylin.

53. The process of claim 1 wherein the composition comprises guanylin.

54. The process of claim 1 wherein the composition comprises lymphoguanylin.

55. The process of claim 1 wherein the composition comprises prolymphoguanylin.

56. The process of claim 1 wherein the composition comprises heat stable enterotoxin.

57. The process of claim 1 wherein the composition comprises a polypeptide, which is degraded with endogenous proteases of the subject, into uroguanylin.

58. The process of claim 1 wherein about 0.5 mg to about 2 mg of the polypeptide is administered per kilogram of the subjects weight.

59. The process of claim 1 wherein the subject is human.

60. The process of claim 1 wherein said peptides are administered in a pharmaceutical composition which contains said peptide and one or more pharmacologically acceptable, inert or physiologically active diluents of adjuvants.

61. The process of claim 1 wherein X₁ is glutamic acid, X₂ is leucine, X₃ is isoleucine, X₄ is valine, X₅ is alanine, X₆ is threonine, and X₇ is glycine.

62. A process for the prevention, inhibition and treatment of cancer in the intestine of a subject, the process comprising administering to the subject the composition of claim 1.

63. A process for the prevention, inhibition and treatment of cancer in the intestine of a subject, the process comprising administering to the subject the composition of claim 2.

64. The process of claim 62 wherein the composition comprises uroguanylin.

65. The process of claim 63 wherein the composition comprises uroguanylin.

66. The process of claim 62 wherein the composition comprises pro-uroguanylin.

67. The process of claim 63 wherein the composition comprises pro-uroguanylin.

68. A process for retarding the development of polyps and prevention, inhibition and treatment of polyps in the intestine of a subject, the process comprising administering to the subject a composition comprising an agonist peptide or compound which binds to a guanylate cyclase receptor GC-C in the intestine of the subject, in combination with a cyclooxygenase-2 inhibitor.
